(19) United States
(12) Patent Application Publication
(10) Pub. No.: US 2015/0011827 A1
(43) Pub. Date: Jan. 8, 2015

(54) MEDICAL TREATMENT INSTRUMENT

(71) Applicant: Terumo Kabushiki Kaisha, Tokyo (JP)

(72) Inventors: Yasushi KINOSHITA, Fujinomiya-shi (JP); Kenichi KUMOYAMA, Fujinomiya-shi (JP)

(21) Appl. No.: 14/497,850

(22) Filed: Sep. 26, 2014

Related U.S. Application Data
(63) Continuation of application No. PCT/JP2013/056535, filed on Mar. 8, 2013.

(30) Foreign Application Priority Data
Mar. 8, 2012 (JP) 2012-072772

(51) Int. Cl. A61B 1/233; A61M 39/22; A61M 25/01; A61M 29/02

(52) U.S. Cl. CPC A61B 1/233; A61M 29/02; A61M 39/221; A61M 25/01
USPC 600/114

(57) ABSTRACT

A medical treatment instrument includes an insertion tube configured to be inserted into a body of a patient; an image information acquisition device configured to be inserted in a withdrawable manner in the insertion tube; and a first conveyance path configured such that an object is conveyable into the insertion tube via the first conveyance path, the first conveyance path being located at an intermediate portion of the insertion tube so as to communicate with the insertion tube. The medical treatment instrument is configured such that the image information acquisition device is retractable to a retracted position proximal of the conveyance path when the object to be conveyed is conveyed through the insertion tube via the conveyance path.

### CROSS-REFERENCE TO RELATED APPLICATIONS

This application is a continuation application filed under 35 U.S.C. 111(a) claiming the benefit under 35 U.S.C. §§120 and 365(c) of PCT International Application No. PCT/JP2013/056535 filed on Mar. 8, 2013, which is based upon and claims the benefit of priority of Japanese Application No. 2012-072772 filed on Mar. 28, 2012, the entire contents of which are hereby incorporated by reference in their entireties.

### BACKGROUND

1. Technical Field

The present disclosure relates to a medical treatment instrument.

2. Background Art

Conventionally, there have been known medical treatment instruments, such as endoscopes and catheters (see, for example, JP-T-2008-526360).

The medical treatment instrument described in JP-T-2008-526360 includes an insertion section having an imaging device and a fluid conveyance lumen, and is so configured that in the state in which the insertion section is inserted in the body of a patient, observation can be performed based on an image captured by the imaging device, and a fluid such as water and a drug can be conveyed into the inside of the body.

However, the medical treatment instrument according to the related art, such as the one described in Patent Document 1, has a configuration in which the lumen for the imaging device and the fluid conveyance lumen are provided in the insertion section independently from each other and in parallel to each other. This configuration has a problem in that the insertion section must be made to be thick (large in diametrical size), making it difficult for the insertion section to be inserted into a living body.

### SUMMARY OF INVENTION

One objective of certain embodiments of the present invention is to provide a medical treatment instrument that can be easily inserted into a living body.

According to one embodiment of the present invention, a medical treatment instrument includes: an insertion tube that is inserted into a body of a patient; an image information acquisition section that is inserted in a withdrawable manner in the insertion tube; and a conveyance path through which to convey an object to be conveyed located within the insertion tube, the conveyance path provided at an intermediate portion of the insertion tube so as to communicate with the insertion tube, in which the image information acquisition section is adapted to be capable of being retracted further toward a proximal side in an insertion direction of the insertion tube than the conveyance path when the object to be conveyed is conveyed by the conveyance path.

In one aspect, the conveyance path is provided in communication with the insertion tube in which the image information acquisition section is inserted in a withdrawable manner. This ensures that the insertion tube as an insertion path for the image information acquisition section can be utilized also as a conveyance path. Therefore, the conveyance path can be integrated with the insertion path for the image information acquisition section, so that the insertion tube can be made thin (small in diametrical size), as compared with the case where the insertion path for the image information acquisition section and the conveyance path are provided in parallel to each other, as in a system according to the related art. Consequently, an embodiment of the medical treatment instrument can be easily inserted into a living body.

In one aspect, the conveyance path is a fluid conveyance path through which to convey a fluid located in the insertion tube, and the medical treatment instrument includes a valve body that seals the proximal side of the insertion tube, the valve body provided on the proximal side of the insertion tube relative to the conveyance path.

In one aspect, the valve body is provided on the proximal side of the insertion tube relative to the conveyance path, which ensures that the proximal side of the insertion tube can be sealed, even in the case of using the conveyance path as a fluid conveyance path. In this instance, since the image information acquisition section is adapted to be capable of being retracted toward the proximal side of the insertion tube, the image information acquisition section can be prevented from blocking the conveyance of a fluid by the conveyance path. Further, since the proximal side of the insertion tube to which the image information acquisition section is retracted communicates with the fluid conveyance path, a fluid can be made to flow in to the proximal side of the insertion tube from the fluid conveyance path, attendant on the conveyance of the fluid; therefore, the image information acquisition section can be cleaned with the fluid.

In one aspect, an embodiment of the present invention includes a position fixture for fixing a retracted position of the image information acquisition section.

In one aspect, the retracted position of the image information acquisition section can be fixed by the position fixture, which eliminates the need for the operator to keep holding the image information acquisition section. Therefore, the burden on the operator can be alleviated, and the operator is allowed to concentrate on diagnosing or treating a patient.

In one aspect, an embodiment of the present invention includes an expansion body that is expanded outward in a radial direction of the insertion tube, the expansion body provided at an outer circumference of the insertion tube.

In one aspect, it is possible, by inserting the part of the expansion body of the insertion tube into a stenosed part in a living body and expanding the expansion body, to expand and treat the stenosed part.

In one aspect, an embodiment of the present invention includes an expansion body that is expanded outward in a radial direction of the insertion tube, the expansion body inserted in a withdrawable manner in the insertion tube from the conveyance path.

In one aspect, the expansion body is inserted via the conveyance path into the insertion tube in a withdrawable manner, which eliminates the need to provide an expansion body at an outer circumference of the insertion tube. Therefore, the insertion tube can be made further thinner (smaller in diametrical size). Consequently, the an embodiment of the medical treatment instrument can be inserted further easily.

In one aspect, an embodiment of the present invention is a sinusitis treatment instrument.

According to an embodiment of the present invention, while the medical treatment instrument is a sinusitis treatment instrument, it has the insertion guide part. This ensures that the medical treatment instrument can be assuredly inserted into a paranasal sinus and into a natural ostium through which the paranasal sinus and a nasal cavity communicate with each other. Accordingly, a treatment necessary for the sinusitis treatment can be carried out without performing a surgical procedure.

### BRIEF DESCRIPTION OF DRAWINGS

FIG. 1 is a side view showing, partly in section, a medical treatment instrument according to an embodiment of the present invention.

FIG. 2A is a partial sectional view of a grasping section of the medical treatment instrument showing multiple positions of a first operating portion.

FIG. 2B is a partial sectional view of the grasping section of the medical treatment instrument shown in FIG. 2A from a different perspective.

FIG. 3 is an exploded perspective view of an insertion tube of the medical treatment instrument.

FIG. 4A is an end-side view showing a valve body of the medical treatment instrument.

FIG. 4B is an end view showing the valve body of the medical treatment instrument.

FIG. 5 is a side-end perspective view showing the configuration in the surroundings of an insertion guide part of the medical treatment instrument.

FIG. 6 is a side view showing, partly in section, a medical treatment instrument according to a second embodiment of the present invention.

### DETAILED DESCRIPTION

Now, embodiments of the present invention will be described below, based on the drawings.

It is to be noted that in a second embodiment and thereafter, component members which are the same as the component members in a first embodiment to be described below and component members which have functions equivalent or similar to the functions of the component members in the first embodiment will be denoted by the same reference symbols as used for the component members in the first embodiment, and descriptions of them will be omitted or simplified.

### First Embodiment

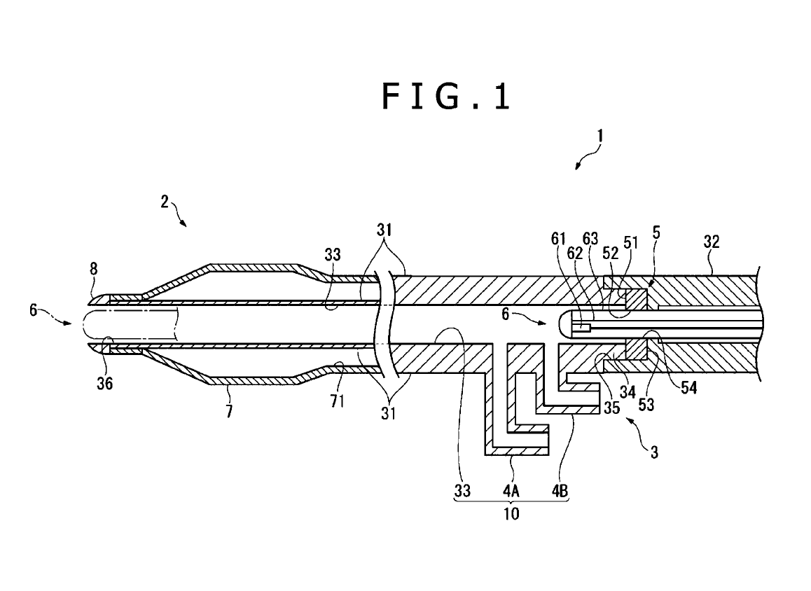

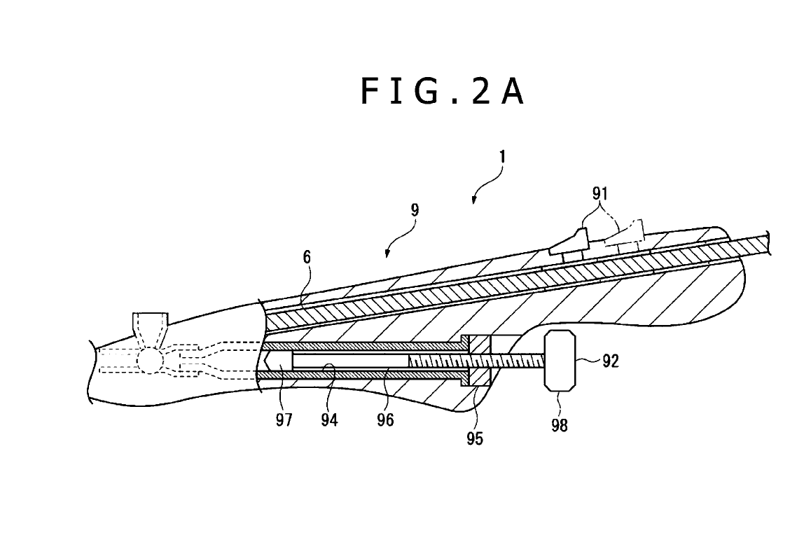

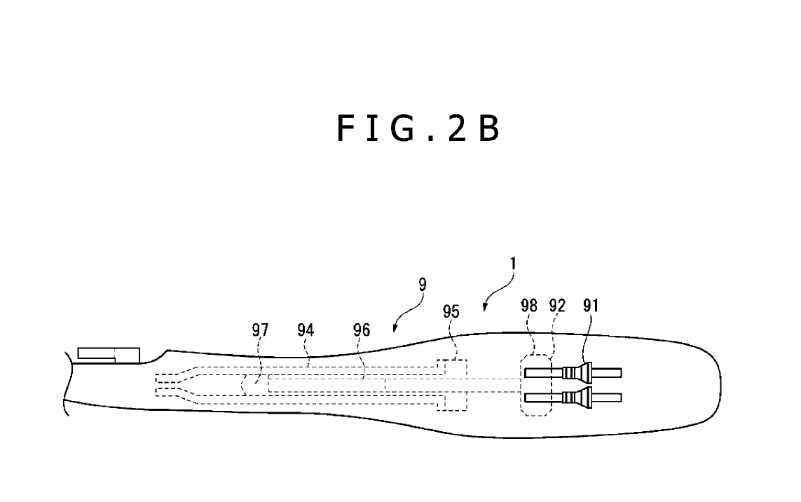

As shown in FIGS. 1, 2A and 2B, a medical treatment instrument 1 according to a first embodiment includes: an insertion section 2 (FIG. 1) that is inserted in a body of a patient; and a grasping section 9 (FIGS. 2A and 2B) that is provided on a proximal side of the insertion section 2.

In FIG. 1, the insertion section 2 includes: an insertion tube 3 as an elongated main body; first and second conveyance paths 4A and 4B as conveyance paths through which to convey an object to be conveyed located within the insertion tube 3, the first and second conveyance paths 4A and 4B being provided at intermediate portions of the insertion tube 3 so as to communicate with the insertion tube 3; a valve body 5 that seals the proximal side of the insertion tube 3, the valve body 5 being provided on the proximal side of the insertion tube 3 relative to the conveyance paths 4A and 4B; an endoscope 6 as an image information acquisition section that acquires an image of the forward side in an insertion direction, the endoscope 6 being inserted in a withdrawable manner in the insertion tube 3; an expansion body 7 that is expanded outward in the radial direction of the insertion tube 3, the expansion body 7 being provided at an outer circumference of the insertion tube 3; and an insertion guide part 8 detachably attached to a distal end of the insertion tube 3.

The insertion tube 3 includes: a distal tube 31 to which the insertion guide part 8 is attached; a proximal tube 32 connected to the grasping portion 9; and a lumen 33 opening toward the insertion guide part 8. The distal tube 31 and the proximal tube 32 are connected to each other in an attachable and detachable manner. The space inside the distal tube 31 and the space inside the proximal tube 32 constitute the lumen 33.

The distal tube 31 is provided with a proximal portion 34 reduced in diameter as compared with the distal side, and is provided with the conveyance paths 4A and 4B which are communicate with the lumen 33. Here, the first conveyance path 4A is a conveyance path through which a fluid as an object to be conveyed is conveyed from the inside of a living body to the outside; specifically, the first conveyance path 4A is so configured that such a fluid as snivel can be sucked therethrough by a suction device (not shown) connected to the first conveyance path 4A. In addition, the second conveyance path 4B is a conveyance path through which a fluid is conveyed into the inside of a living body from the outside; specifically, the second conveyance path 4B is so configured that such a fluid as washing water can be conveyed into the inside of the living body by means of a pump (not shown) connected to the second conveyance path 4B. The first conveyance path 4A, the second conveyance path 4B, and the lumen 33 constitute a fluid conveyance path 10. With the conveyance paths 4A and 4B thus provided in communication with the insertion tube 3, the fluid conveyance path 10 is integrated with the lumen 33 that serves as an insertion path for the endoscope 6.

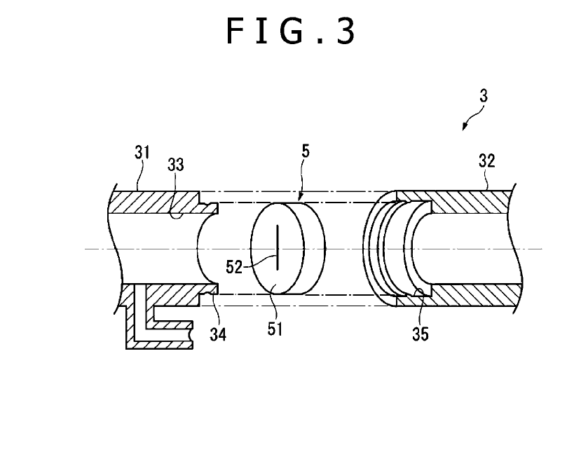

As shown in FIG. 3, also, the proximal tube 32 is provided, at an end face on the distal side thereof, with a recess 35 greater in inside diameter than the lumen 33, and the valve body 5 is accommodated in the recess 35. In a state wherein the valve body 5 is accommodated in the recess 35, the proximal portion 34 of the distal tube 31 is fitted in the recess 35, whereby the distal tube 31 and the proximal tube 32 are fixed to each other.

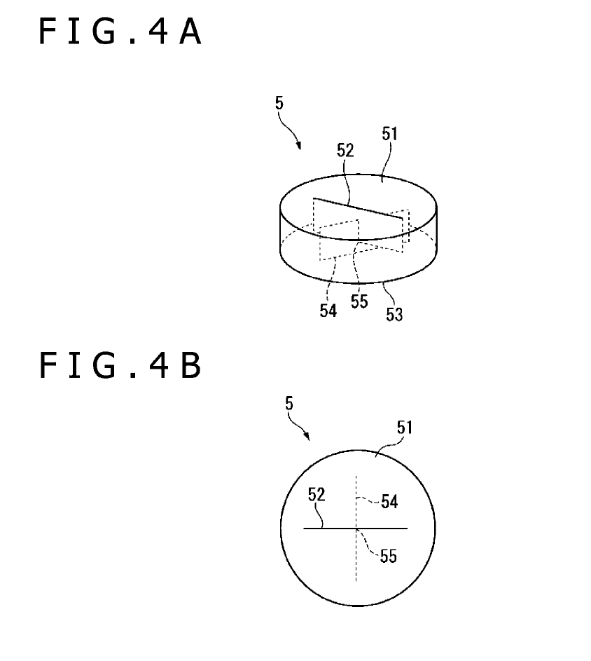

The valve body 5 is configured by use of an elastic material such as rubber or resin. As shown in FIGS. 4A and 4B, the valve body 5 is provided with a first cut 52 formed in an end face 51 on a first side, and with a second cut 54 formed also in an end face 53 on a second side. The first cut 52 and the second cut 54 intersect each other at an intersection portion 55 inside the valve body 5. This configuration enables the valve body 5 to permit insertion and passage of the endoscope 6 through the first cut 52, the intersection portion 55, and the second cut 54 under elastic deformation of the valve body 5, and to hold liquid-tight the lumen 33 of the insertion tube 3. It is to be noted that the intersection angle between the first and second cuts 52 and 54 may not necessarily be a right angle; in other words, the first and second cuts 52 and 54 may intersect each other obliquely.

The endoscope 6 includes: an imaging section 61 having an imaging element such as a CCD (Charge Coupled Device) image sensor; a light transmission section 62, such as an optical fiber, through which to transmit light toward a distal portion of the endoscope 6; and an outer tube 63 in which to accommodate the imaging section 61 and the light transmission section 62. The endoscope 6 may be configured to be able to be curved by operating means such as wire (not shown) connected to a distal portion of the endoscope 6. The imaging section 61 is not restricted to this one configuration; for example, the imaging section 61 may be a digital video camera that uses other imaging element such as a CMOS (Complementary Metal Oxide Semiconductor) image sensor, an image fiber that acquires and transmits images by utilizing optical fibers, or an imaging system that transmits images by an optical system including an objective lens and a plurality of relay lenses.

The expansion body 7 is configured by using a flexible material such as a polymer. The inside of the expansion body 7 communicates with a flow path 71 provided in the periphery of the insertion tube 3. With a fluid introduced into the expansion body 7 via the flow path 71, the expansion body 7 is expanded radially.

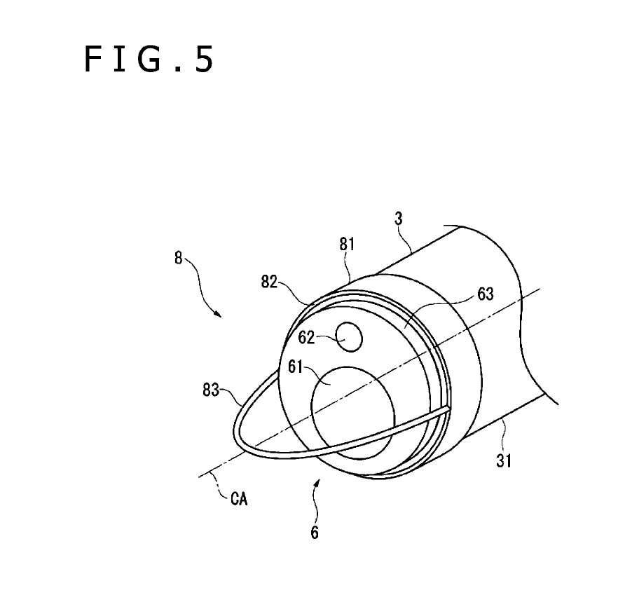

As shown in FIG. 5, the insertion guide part 8 is formed to be gradually decreased in diameter along the distal direction. Specifically, the insertion guide part 8 includes: a frame body 81 that is attached to a distal portion of the insertion tube 3; and a bridge portion 83 bridgingly provided between points of an end edge 82 of the frame body 81 in such a manner as to project toward the distal side of the insertion tube 3. The bridge portion 83 is composed of a single linear body which is flexible. The bridge portion 83 is bridgingly provided, in a curved shape, between two end points which are located symmetrically with respect to the center axis CA of the frame body 81. The insertion guide part 8, particularly the bridge portion 83, is configured by use of an elastic material, preferably an elastic metallic material; for example, it is preferably configured by use of a shape memory alloy that shows superelasticity in a use state. Such a shape memory alloy is preferably concretely composed of a nickel-titanium alloy. Besides, in FIG. 1, the positional relationship between the insertion guide part 8 and the insertion tube 3 is omitted. The insertion guide part 8 may be attached to an outer circumferential surface of a distal end of the insertion tube 3, or may be attached to an inner circumferential surface of the distal end. Further, the insertion guide part 8 may be embedded in the inside of the insertion tube 3, or may be firmly attached in the manner of being clamped between an outer circumferential surface of the insertion tube 3 and other member. The method for firmly attaching the insertion guide part 8 is not specifically restricted. The insertion guide part 8 may be firmly attached by an adhesive, brazing or the like, may be soldered, or may be firmly attached by caulking.

As shown in FIGS. 2A and 2B, the grasping section 9 includes: a first operating portion 91 as a position fixture for fixing an advanced/retracted position of the endoscope 6 inside the insertion tube 3; and a second operating portion 92 for expanding or contracting the expansion body 7 and for maintaining an expanded state of the expansion body 7.

The first operating portion 91 holds the endoscope 6 inside the grasping section 9. The first operating portion 91 is provided to be slidable toward the insertion tube 3, and is configured to be fixable at a position indicated by solid line and a position indicated by alternate long and short dash line in FIG. 2A. Specifically, when the first operating portion 91 is located in the position of solid line, the endoscope 6 has been inserted to such an extent that the imaging section 61 reaches the insertion guide part 8; on the other hand, when the first operating portion 91 is located in the position of alternate long and short dash line, the endoscope 6 has been retracted to the proximal side of the insertion tube 3 relative to the conveyance paths 4A and 4B and is fixed in the retracted position.

The second operating portion 92 is provided in an advanceable and retractable manner in a fluid introduction path 94 communicating with the flow path 71 for the expansion body 7, and is so configured that the expansion body 7 is expanded or contracted when the second operating portion 92 is advanced or retracted. Specifically, the second operating portion 92 includes: a rod 96 put into screw engagement with a threaded hole formed in a plugging member 95 for plugging the fluid introduction path 94; a sealing member 97 which is provided on one end side of the rod 96 inside the fluid introduction path 94 and which seals the fluid introduction path 94; and a knob part 98 provided on the other end side of the rod 96 in the outside of the fluid introduction path 94.

Now, the use procedure and operation of the medical treatment instrument 1 when the medical treatment instrument 1 is used as a sinusitis treatment instrument, as an example of use of the medical treatment instrument 1, will be described below.

First, in a state in which the endoscope 6 has been inserted to the distal end of the insertion section 2, the operator inserts the insertion section 2 into a nostril. Here, since the fluid conveyance path 10 is integrated with the lumen 33 of the insertion tube 3, the insertion tube 3 is thin (small in diametrical size), as compared with the case where an insertion path for the endoscope 6 and the fluid conveyance path 10 are provided in parallel to each other. Therefore, the insertion section 2 as a whole can be made thinner, so that the insertion section 2 can be inserted easily.

Further, the insertion guide part 8 guides the insertion section 2 while forcing open the insertion route toward the outer circumference of the insertion tube 3 by the bridge portion 83. In this instance, since the bridge portion 83 is configured by use of a linear body, its thickness size as viewed from the insertion direction is small, so that the resistance at the time of insertion can be suppressed. In addition, since the bridge portion 83 is flexible, it can follow the shape of the insertion route through deformation. Therefore, the ability of the insertion guide part 8 to pass the insertion route can be enhanced, and, accordingly, the insertion section 2 can be inserted more easily. Furthermore, since the bridge portion 83 is bridgingly provided in a curved shape, the bridge portion 83 would not locally press the inside of the living body, so that it can be prevented from damaging the tissue inside the nasal cavity.

Meanwhile, the operator inserts the insertion section 2 while checking the conditions in the insertion route, based on an image acquired by the endoscope 6. Here, since the bridge portion 83 is composed of a linear body and the area of projection of the bridge portion 83 in the insertion direction is small, the bridge portion 83 can be prevented from obstructing the viewing of the image acquired by the endoscope 6. In addition, even where a sticky matter or solid matter is present in the insertion route, the bridge portion 83 can split such a matter into pieces, so that adhesion of the sticky matter or solid matter to the bridge portion 83 can be restrained. Consequently, a sufficient field of view for imaging by the endoscope 6 can be secured.

When the expansion body 7 of the insertion section 2 inserted is guided to a natural ostium of a paranasal sinus stenosed due to sinusitis, the operator can operate the second operating portion 92 so as to introduce a fluid into the expansion body 7 and expand the expansion body 7, thereby expanding and treating the stenosed part of the natural ostium. It is to be noted that when the insertion section 2 is slightly retracted after contraction of the expansion body 7, it is possible to confirm that the once stenosed part has been expanded, based on the image acquired by the endoscope 6.

Thereafter, the operator inserts the insertion section 2 through the expanded natural ostium into the paranasal sinus, whereon it is possible to check the conditions inside the paranasal sinus, based on an image acquired by means of the endoscope 6. In the case where a fluid (such as snivel) or sticky matter is accumulated in the paranasal sinus, the first operating portion 91 is operated so as to retract the endoscope 6 in the insertion tube 3 to the proximal side relative to the conveyance paths 4A and 4B, after which the fluid or sticky matter can be sucked through the lumen 33 of the insertion tube 3 and the first conveyance path 4A. In this instance, since the endoscope 6 has been retracted and the thickness size of the bridge portion 83 as viewed from the insertion direction is small, the endoscope 6 and the bridge portion 83 can be prevented from blocking the suction; accordingly, the suction can be carried out rapidly and assuredly.

On the other hand, cleaning of the inside of the paranasal sinus with a washing fluid such as physiological salt solution can also be performed, by introducing the washing fluid into the second conveyance path 4B. Attendant on this, the washing fluid flows in the insertion tube 3 to the proximal side relative to the conveyance paths 4A and 4B, so that cleaning of the imaging section 61 of the endoscope 6 in the retracted state can be conducted simultaneously with the cleaning of the inside of the paranasal sinus. In this instance, like in the case of the suction mentioned above, the endoscope 6 and the bridge portion 83 can be prevented from blocking the suction, so that the cleaning can be carried out effectively.

Since the medical treatment instrument 1 is provided with the conveyance paths 4A and 4B in communication with the insertion tube 3 in which the endoscope 6 is inserted in a withdrawable manner, the insertion tube 3 as the insertion path for the endoscope 6 can be utilized also as the fluid conveyance path 10. Therefore, the fluid conveyance path 10 can be integrated with the insertion path for the endoscope 6. Accordingly, the insertion tube 3 can be made thin (small in diametrical size), as compared with the case where the insertion path for the endoscope 6 and the fluid conveyance path 10 are provided in parallel to each other, as in a system according to the related art. Consequently, the medical treatment instrument 1 can be easily inserted into a living body.

In addition, since the valve body 5 is provided on the proximal side of the insertion tube 3 relative to the conveyance paths 4A and 4B, the proximal side of the insertion tube 3 can be sealed even in the case of using the conveyance paths 4A and 4B as the fluid conveyance path 10. In this instance, since the endoscope 6 can be retracted toward the proximal side of the insertion tube 3, the endoscope 6 can be prevented from blocking the conveyance of the fluid by the fluid conveyance path 10. Further, since the proximal side of the insertion tube 3 to which the endoscope 6 is retracted communicates with the conveyance paths 4A and 4B, the fluid can be made to flow in to the proximal side of the insertion tube 3 via the fluid conveyance path 10, attendant on the conveyance of the fluid; therefore, the endoscope 6 can be cleaned with the fluid.

Furthermore, since the retracted position of the endoscope 6 can be fixed by the first operating portion 91, it is unnecessary for the operator to keep holding the endoscope 6. Therefore, the burden on the operator can be alleviated. Consequently, the operator is allowed to concentrate on diagnosing or treating the patient.

Moreover, by inserting the part of the expansion body 7 of the insertion tube 3 into a stenosed part in a living body and expanding the expansion body 7 in the inserted state, the stenosed part can be expanded and treated.

### Second Embodiment

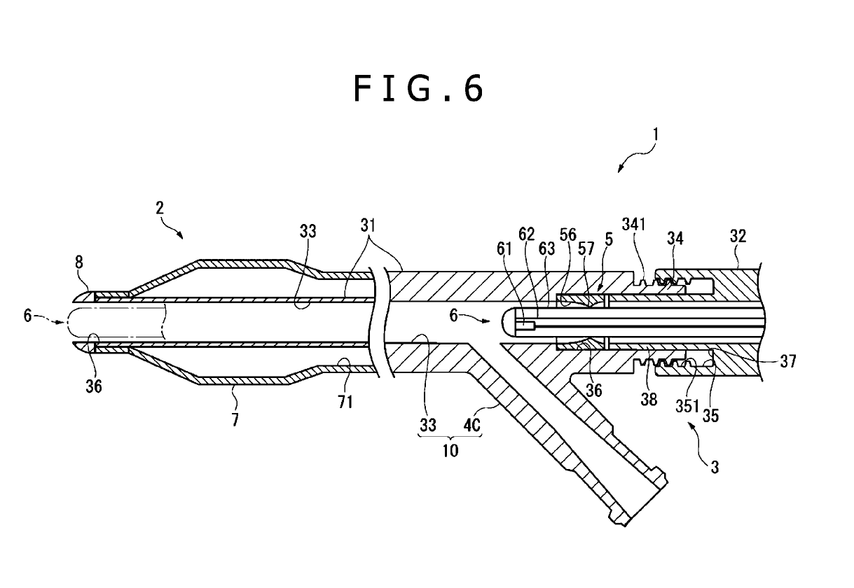

Now, a second embodiment of the present invention will be described below, based on FIG. 6.

As shown in FIG. 6, a medical treatment instrument 1 according to this embodiment differs from that in the first embodiment in the shapes of the distal tube 31 and the proximal tube 32 and in the shape of the valve body 5.

At an intermediate portion of a distal tube 31, a conveyance path 4C branched from the distal tube 31 in a Y shape is provided in communication with a lumen 33. A proximal portion 34 of the distal tube 31 is formed at its outer circumference with a male screw 341. In addition, the proximal portion 34 is provided at an end face thereof with a recess 36 greater in inside diameter than the lumen 33. A valve body 5 is accommodated in the recess 36.

A recess 35 of a proximal tube 32 is formed at its inner circumference with a female screw 351 for screw engagement with the male screw 341 of the distal tube 31. In addition, at the recess 35, there is provided a projecting portion 38 which projects in the axial direction of the proximal tube 32 from a bottom surface of the recess 35.

The valve body 5 is configured by use of an elastic material such as rubber or resin. The valve body 5 is provided with a penetrating path 56 penetrating therethrough from an end face 51 on one side to an end face 53 on the other side, and with a diametrically reduced portion 57 where the diameter of the penetrating path 56 is partly reduced.

When the distal tube 31 and the proximal tube 32 in the medical treatment instrument 1 are rotated relative to each other, starting from the state as shown in FIG. 6, to thereby bring the distal tube 31 and the proximal tube 32 toward each other along the axial direction, the valve body 5 is pushed in the axial direction by the projecting portion 38 of the proximal tube 32. This results in that the valve body 5 is deformed, the penetrating path 56 as a whole is reduced in diameter, and the lumen 33 of the insertion tube 3 is held liquid-tight by the diametrically reduced portion 57 of the penetrating path 56.

According to this embodiment as above, the equivalent or similar effects to those of the first embodiment can be obtained.

It is to be noted that the present invention is not to be restricted to the aforementioned embodiments, and the invention includes those modifications, improvements and the like which are within such a scope that the object of the invention can be achieved.

For instance, while the insertion guide part 8 is provided at the distal end of the insertion tube 3 for accommodating the endoscope 6 in the above embodiments, this is not restrictive. The insertion guide part 8 can be attached to any part of the medical treatment instrument insofar as the part is in a distal portion inserted into a living body. For example, the insertion guide part 8 can be used also for a medical treatment instrument such as an endoscope, a guiding catheter, a balloon catheter, etc. In addition, the sectional shape of the insertion guide part 8 and the sectional shape of the elongated body to which the insertion guide part 8 is attached are not restricted to a true circle, and may, for example, be an ellipse, a polygon, or the like. Besides, the insertion guide part 8 may not necessarily be provided.

In addition, while the insertion guide part 8 is provided with the single bridge portion 83 in the above embodiments, a plurality of bridge portions 83 may be provided at different positions of the end edge 82 of the frame body 81.

Besides, while the bridge portion 83 is composed of a linear body in the above embodiment, the bridge portion 83 may be composed of a band-shaped body, in which case an end portion in the width direction of the band-shaped body may project toward the distal side of the insertion tube 3. Furthermore, a bridge portion 83 composed of a linear body and a bridge portion 83 composed of a band-shaped body may be provided in combination.

While the expansion body 7 is provided at an outer circumference of the insertion tube 3 in the above embodiments, the medical treatment instrument 1 may be configured without providing any such expansion body 7. Specifically, a configuration may be adopted in which the medical treatment instrument 1 includes a balloon catheter having an expansion body, and the balloon catheter can be inserted into and withdrawn from the lumen 33 via the first conveyance path 4A or the second conveyance path 4B. Thus, it is not indispensable that the conveyance path in the insertion section 2 be configured as the fluid conveyance path 10; in other words, the conveyance path in the insertion section 2 may be configured as a conveyance path through which to convey such an object to be conveyed as a balloon catheter or a guide wire.

While the retracted position of the endoscope 6 in the insertion tube 3 can be fixed by the first operation section 91 in the above embodiments, this is not restrictive of the means of fixing the retracted position of the endoscope 6. For instance, of the endoscope 6, the distal portion where the imaging section 61 is provided may be enlarged in diameter, or a portion on the proximal side may be set smaller than the distal portion in diameter, whereby movement of the distal portion can be restricted by the valve body 5, and the retracted position of the endoscope 6 can be fixed.

While the medical treatment instrument 1 is used for observation of the inside of a paranasal sinus or treatment of sinusitis in the above embodiments, the medical treatment instrument 1 may be used for observation or treatment of other part in a living body.

The present invention is applicable not only to medical treatment instruments for diagnosis or therapy of a paranasal sinus but also to medical treatment instruments for other diagnoses or therapies that are not attended by a surgical procedure.

What is claimed is:

1. A medical treatment instrument comprising:
an insertion tube configured to be inserted into a body of a patient;
an image information acquisition device configured to be inserted in a withdrawable manner in the insertion tube; and
a first conveyance path configured such that an object is conveyable into the insertion tube via the first conveyance path, the first conveyance path being located at an intermediate portion of the insertion tube so as to communicate with the insertion tube,
wherein the medical treatment instrument is configured such that the image information acquisition device is retractable to a retracted position proximal of the conveyance path when the object to be conveyed is conveyed through the insertion tube via the conveyance path.

2. The medical treatment instrument according to claim 1, wherein the conveyance path is a fluid conveyance path through which a fluid is conveyable into the insertion tube.

3. The medical treatment instrument according to claim 1, further comprising a valve body configured to seal a proximal side of the insertion tube, the valve body being located proximal of the conveyance path.

4. The medical treatment instrument according to claim 3, wherein the valve body is configured such that the image information acquisition device is insertable into and passable through the valve body.

5. The medical treatment instrument according to claim 1, further comprising a first operating portion configured to fix the image information acquisition section in the retracted position.

6. The medical treatment instrument according to claim 1, further comprising an expansion body configured to be expanded outward in a radial direction of the insertion tube, the expansion body provided at an outer circumference of the insertion tube.

7. The medical treatment instrument according to claim 6, further comprising a second operating portion configured to communicate with a fluid in a flow path which is in communication with the expansion body such that, when the second operating portion is advance or retracted, the expansion body is expanded or contracted.

8. The medical treatment instrument according to claim 1, further comprising an expansion body that is expanded outward in a radial direction of the insertion tube, the expansion body being insertable in a withdrawable manner into the insertion tube via the conveyance path.

9. The medical treatment instrument according to claim 1, wherein the medical treatment instrument is configured for use in sinusitis treatment.

10. The medical treatment instrument according to claim 1, further comprising a second conveyance path configured such that an object is conveyable into the insertion tube via the second conveyance path, the second conveyance path being located at an intermediate portion of the insertion tube so as to communicate with the insertion tube.

11. The medical treatment instrument according to claim 10, wherein the first conveyance path is configured such that a fluid can be sucked therethrough by a suction device connected to the first conveyance path.

12. The medical treatment instrument according to claim 11 wherein the second conveyance path is configured such that a fluid can be conveyed into an inside of a body by means of a pump connected to the second conveyance path.

13. The medical treatment instrument according to claim 1, wherein the image information acquisition device is an endoscope.

14. The medical treatment instrument according to claim 1, further comprising an insertion guide part.

15. The medical treatment instrument according to claim 14, wherein the insertion guide part comprises a frame body attached to a distal portion of the insertion tube, and a bridge portion.
